(19) United States
(12) Patent Application Publication
(10) Pub. No.: US 2025/0041493 A1
(43) Pub. Date: Feb. 6, 2025

(54) CONNECTOR AND METHOD OF SECURING A TUBE

(71) Applicant: Mayborn (UK) Limited, Newcastle Upon Tyne (GB)

(72) Inventors: Matthew Gossington, Newcastle Upon Tyne (GB); Robert Kenneth Alexander Jervis, Newcastle Upon Tyne (GB)

(21) Appl. No.: 18/716,656

(22) PCT Filed: Dec. 9, 2022
(86) PCT No.: PCT/GB2022/053144

§ 371 Date: Jun. 5, 2024

(30) Foreign Application Priority Data
Dec. 10, 2021 (GB) 2117909.8

(51) Int. Cl. A61M 1/06; A61M 39/10

(52) U.S. Cl. CPC A61M 1/062; A61M 39/1011; A61M 2207/00

(57) ABSTRACT

The present invention relates to connector, in particular a connector suitable for securing to a breast pump element. A method of securing a tube portion to a breast pump element is also disclosed. The connector comprises: a tube portion with a first opening; and a securing member, mounted to the first opening, and configured to be engageable with a breast pump element having a fluid port so as to enclose the fluid port and form a fluid flow path between the first opening and the breast pump element. The securing member is configured to resiliently deform to be urged into a sealing abutment with the breast pump element when negative pressure is applied within the tube portion.

The present invention relates to connector, in particular a connector suitable for securing to a breast pump element. A method of securing a tube portion to a breast pump element is also disclosed.

### Introduction

Breast pumps are used by nursing mothers to allow them to conveniently express their breast milk for storage and later feeding to infants. Such breast pumps stimulate milk expression by mimicking the action of a baby by applying suction to the breast. Negative pressure, that is suction, is applied to the breast in short or cyclical pulses in order to provide efficient milk expression.

In use, a breast contacting part of a breast pump, such as a horn or massaging portion, is positioned onto the breast so that at least the nipple is received within a narrowed opening or recess within the breast contacting part. Once positioned, a negative pressure source in the breast pump is activated to apply negative pressure to the breast via the narrowed opening or recess, thereby encouraging expression of breast milk. A breast pump thereby delivers negative pressure to the breast contacting part. In this way, breast pump components, or elements, are provided to deliver, or convey, a negative pressure from a negative pressure source to the user's breast.

In order to ensure hygiene, certain elements of breast pumps must be cleaned and potentially sterilised between uses. In this way, breast pumps typically include detachable or modular elements which are readily taken apart for storage or cleaning or, in the case of containers, storage of milk. In certain breast pumps, flexible diaphragms are used to isolate tubes, conduits and volumes while transmitting the negative pressure from the pump to the breast.

A problem with known breast pumps is that it can be awkward to dissemble and assemble elements of the breast pump assembly. In particular, incorrect or inaccurate coupling of connectors, tubes, fluid ports or the like to breast pump elements can result in unreliable sealing between the elements, which reduces the efficiency of the negative pressure. Such arrangements require additional assembly time for the user. They may also be difficult to align and seal, for example by inserting a narrow tube into a small opening at a specific orientation. Other known arrangements rely on components to clamp together the assembled elements, to ensure a seal therebetween, also requiring additional assembly time for the user.

In addition, breast pump elements may need to be oriented and moved towards each other in a specific direction, for example a linear movement of axially-aligned connector and port, in order to ensure that one part is effectively received within the other. Furthermore, certain connector and ports may only form reliable seals if they are oriented to approach one another at a specific angle, for example a connector should approach a port perpendicular to the opening of the port.

A further known problem is that repeated assembly and disassembly of breast pump elements may produce wear or distortion between elements, thereby leading to seal failure after prolonged use. Even when elements are assembled without leaks, seals may be susceptible to relative movement between the engaged elements. The movement of the breast pump during use, for example, causes leaks, or at least temporary leakage, due to the failure of seals between interconnecting elements. Any leaks will reduce the efficiency of the negative pressure source and may necessitate replacing elements.

Accordingly, it would be useful to provide a connector for a breast pump which overcomes one or more of the above problems. That is, it would be useful to provide a connector for easily and reliably securing a connector to a breast pump element. In particular, it would be useful to provide a connector that may be easily aligned by a user or that may be aligned in a range of orientations while still providing a reliable seal.

Further, it would also be useful to provide a connector which is wear resistant.

It would also be useful to provide a connector which provides a robust seal, particularly a connector which provides a seal that overcomes distortion of, or relative movement of, the connector and breast pump elements forming the seal. A robust seal also is resistant to contamination, for example by ingress of dirt between the connector and breast pump elements.

It would also be useful to provide a method of connecting a connector or tube portion to a breast pump element which addresses the problems described above.

### SUMMARY OF THE INVENTION

According to an aspect of the invention, there is provided a connector for securing a tube portion to a breast pump element. That is, there is provided a breast pump element connector for securing a tube portion.

According to an aspect of the invention, there is provided a breast pump assembly including a negative pressure source and a connector fluidly connected to the negative pressure source, wherein the connector is configured for securing a tube portion to a breast pump element.

According to an aspect of the invention, there is provided a method of securing a tube portion to a breast pump element.

As used herein, a breast pump element may include one or more components of the breast pump provided in fluid communication with a negative pressure source so as to direct or convey negative pressure to the user's breast. A non-exhaustive list of exemplary breast pump elements may include, one, or an assembly including more than one, of: a pump housing, a milk storage container, a container cover or cover assembly, a horn, a horn insert, a horn housing, a diaphragm or a diaphragm assembly.

According to a further aspect of the invention, there is provided a connector suitable for securing to a breast pump element, the connector including or comprising:
a tube portion with a first opening;
and a securing member, mounted to the first opening, and configured to be engageable with a breast pump element having a fluid port so as to enclose the fluid port and form a fluid flow path between the first opening and the breast pump element; and
wherein the securing member is configured to resiliently deform to be urged into a sealing abutment with the breast pump element when negative pressure is applied within the tube portion.

Aptly, the connector is a breast pump element connector.

Aptly, by enclosing the fluid port, the connector covers the fluid port.

Aptly, the securing member may include an annular flange. The annular flange may, for example, extend from the first opening.

Aptly, the tube portion may have an axis, particularly a central axis extending normal to the first opening and the annular flange extends outwardly and axially from the first opening. Stated differently, the annular flange may flare outwardly and away from the first opening.

Aptly, the annular flange may be frustoconical.

Aptly, the annular flange may have a thickness in a range of from 1 mm to 3 mm, preferably in the range of from 1.2 mm to 2.0 mm.

Aptly, the connector may include an elastomer. More aptly, the elastomer may be a silicone rubber or a thermoplastic elastomer. Yet more aptly, the elastomer may have a hardness in the range Shore A 20 to 50, preferably in the range Shore A 30 to 40.

Aptly, the sealing abutment may provide an area of contact with the breast pump element. The area of contact may increase as the securing element is resiliently deformed.

Aptly, the area of contact may be annular. Aptly, the area of contact area when providing a sealing abutment may have a width, wherein the width is in a range of from 2 mm to 5 mm and, preferably, in a range of from 2 mm to 3 mm.

Aptly, the annular flange may be configured so that a perimeter portion of the annular flange deforms outwardly as the securing element is resiliently deformed.

Aptly, the securing member may be provided a first position, wherein the securing member engages a beast pump element and encloses the fluid port and wherein, when negative pressure is applied within the tube portion, the securing member is urged into a second position to provide a sealing abutment with the breast pump element.

Aptly, in moving from the first position to the second position the annular flange, typically at least an outer portion of the annular flange, of the securing element may be deformed outwardly. In this way, a perimeter portion of the annular flange deforms outwardly as the securing element moves to a second position.

Aptly, in moving from the first position to the second position the annular flange, typically at least an outer portion of the annular flange, of the securing element may be deformed to become more flared.

Aptly, in the first position, the annular flange, typically at least an outer portion of the annular flange, may be frustoconical and, in the second position, the annular flange, typically at least an outer portion of the annular flange, extends substantially radially.

Aptly, in moving from the first position to the second position the annular flange, typically at least an outer portion of the annular flange, of the securing element may be deformed by contracting or collapsing, so as to, for example, deform around a projection or projecting feature on the cover assembly.

Aptly, the securing element may include a folded portion or a bellows configured such that, in moving from the first position to the second position, one or more folds of the folded portion or bellows is resiliently deformed.

Aptly, in moving from the first position towards the second position the sealing abutment may provide an increased area of contact between the securing element and the breast pump element. More aptly, the area of contact between the securing element and the cover assembly may increase to a radial width of at least 2 mm. That is, the radial width of annular contact area may increase by at least 2 mm.

Aptly, the tube portion and the securing member may be integrally formed.

Aptly, the connector may include a mounting means to mount the connector within a breast pump assembly.

Aptly, the securing element may be configured to engage with a locating means provided on a breast pump element.

Aptly, the tube portion may be configured to be fluidly connected to a negative pressure source of a breast pump assembly.

According to a further aspect of the invention, there is provided a breast pump assembly including a negative pressure source and a connector suitable for securing to a breast pump element, wherein the connector is mounted to the breast pump assembly and fluidly connected to the negative pressure source, and wherein the connector includes:
a tube portion with a first opening;
and a securing member, mounted to the first opening, and configured to be engageable with a breast pump element having a fluid port so as to enclose the fluid port and form a fluid flow path between the first opening and the breast pump element; and wherein the securing member is configured to resiliently deform to be urged into a sealing abutment with the breast pump element when negative pressure is applied within the tube portion.

Aptly, the connector may be mounted so as to engage a breast pump element releasably secured to the breast pump assembly.

Aptly, the connector may include a mounting means to mount the tube portion in a fixed position relative to the breast pump assembly.

Aptly, the mounting means may include a projection or protrusion on an outer wall of the tube portion. More aptly, the mounting means may be configured to inhibit axial or rotational movement relative to the breast pump assembly.

Aptly, the projection or protrusion may be configured to locate with a retainer provided in the breast pump assembly.

Aptly, the breast pump assembly may be configured to be releasably secured to a breast pump element by relative rotation of the breast pump assembly.

Aptly, the breast pump assembly may be releasably secured to a breast pump element using a bayonet fitting.

Aptly, the securing member may be engaged with a breast pump element so as to enclose a fluid port provided thereon and form a fluid flow path between the first opening and the breast pump element.

Aptly, the breast pump element may include a polished surface for engaging the securing the member.

According to a yet further aspect of the invention, there is provided a method of securing a tube portion to a breast pump element, the method including the steps of:
mounting a securing member to a first opening of a tube portion;
engaging the securing member with a breast pump element having a fluid port provided thereon such that the securing member encloses the fluid port to form a fluid flow path between the first opening and the fluid port; and
applying a negative fluid pressure within the tube portion so as to resiliently deform the securing member and urge the securing member into a sealing abutment with the breast pump element.

Aptly, the step of engaging the securing member with a breast pump element may further include engaging the securing member with a locating means provided on a breast pump element.

Aptly, the step of engaging the securing member with a breast pump element may include covering the fluid port with the securing member.

Aptly, the locating means may include a recess.

Certain examples provide a connector which enables a breast pump to be easily assembled and disassembled. Additionally, certain examples enable a connector to be easily aligned with a breast pump element so as to be engaged with the breast pump element and form a fluid flow path therebetween.

Certain examples provide a connector which engages a breast pump element by any relative movement between the two elements. That is, the elements may be brought into engagement for a sealing abutment from a range of orientations and directions, for example by sliding, rotating or twisting relative to one another.

Certain examples provide a connector which engages a breast pump element to form a sealing abutment at a range of orientations or angle of approach.

Certain examples provide a connector which is wear resistant. That is, certain examples ensure a reliable seal with a breast pump element over prolonged use. In this way, certain examples provide a robust seal to a breast pump element which withstands distortion or relative movement between elements. Alternatively, or additionally, a seal is provided which overcomes contamination, such as by ingress of dirt between elements or on surfaces which engage to form the seal.

Certain examples provide a method of securing a connector or tube portion to a breast pump element with one or more of the above advantages.

### BRIEF DESCRIPTION OF THE DRAWINGS

Embodiments of the invention are now described, by way of example only, hereinafter with reference to the accompanying drawings, in which:

FIG. 1 shows (a) a perspective view and (b) a cross-sectional view of a connector 100 according to an aspect of the invention;

FIG. 2 shows an exploded perspective view of a breast pump assembly including the connector of FIG. 1;

FIG. 3 shows a lower view of the housing and horn of the breast pump assembly of FIG. 2;

FIG. 4 shows a lower perspective view of the housing of the breast pump assembly of FIG. 2;

FIG. 5 shows a perspective view of an alternative cover assembly of a breast pump assembly;

FIG. 6 shows a perspective view of the cover assembly and connector of the breast pump assembly of FIG. 5;

FIG. 7 shows a cross sectional view of certain aspects of the breast pump assembly of FIG. 5 in a first position; and

FIG. 8 shows a cross sectional view of the aspects of FIG. 7 in a second position.

In the drawings, like reference numerals refer to like parts.

### DETAILED DESCRIPTION

Certain terminology is used in the following description for convenience only and is not limiting. The words ‘lower’ and ‘upper’ designate directions in the drawings to which reference is made and are with respect to the described component when assembled and mounted. The words ‘inner’ and ‘outer’ refer to directions toward and away from, respectively, a designated centreline or a geometric centre of an element being described (e.g. central axis), the particular meaning being readily apparent from the context of the description.

Further, as used herein, the terms ‘connected’ and ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

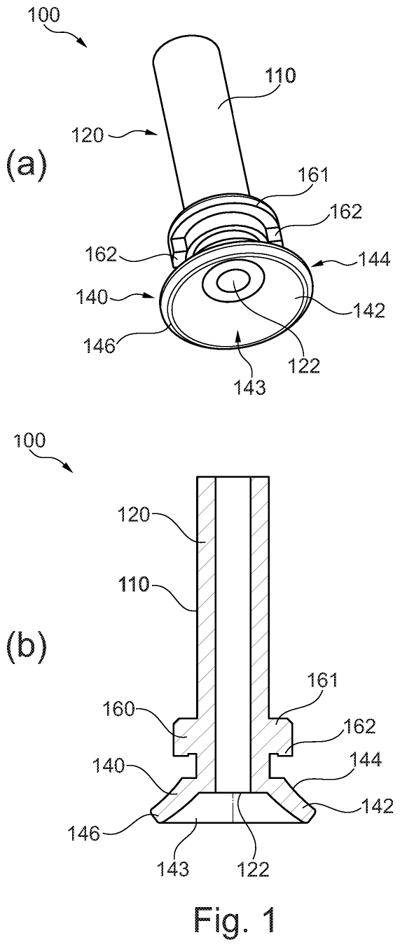

Referring now to FIGS. 1(a) and 1(b) there is shown a connector 100 for securing to a breast pump element. The connector 100 includes a tube portion 120 and a securing member 140. The tube portion 120 includes an outer wall 110 and a first opening 122. The securing member 140 is mounted to the first opening 122. The securing member 140 is configured to be engageable with a breast pump element having a fluid port so as to enclose the fluid port and form a fluid flow path between the first opening and the breast pump element. The securing member 140 is configured to resiliently deform to be urged into a sealing abutment with the breast pump element when negative pressure is applied within the tube portion 120.

The securing member 140 includes an annular flange 142 extending from the first opening 122. That is an annular flange 142 is provided around the first opening 122 and extends away therefrom.

In the example shown, the first opening 122 of the tube portion 120 is annular and the annular flange 142 is extends circumferentially from the opening.

The annular flange 142 is frustoconical. The tube portion 120 has an axis extending normal to the first opening 122. In this way, the annular flange 142 extends outwardly and axially from the first opening 122. Stated differently, the annular flange 142 flares outwardly and away from the first opening 122.

The annular flange 142 has a thickness. The thickness of the annular flange 142 corresponds to a distance between an inner surface 143 and an outer surface 144 of the annular flange 142. In the example shown in FIG. 1, the annular flange 142 has a thickness of 1.3 mm at its outer edge.

The tube portion 120 and the securing member 140 are integrally formed. That is, the connector 100 is formed as a single, unitary part. In the example shown, the connector 100 is formed by injection moulding so that the securing member 140 is provide integral with the tube portion 120.

The connector 100 is formed of an elastomer, in this case, a silicone rubber. The silicone rubber has a hardness of Shore A 30.

The connector 100 includes a mounting means 160 to mount the connector 100 within a breast pump assembly. In this way, the connector 100 may be releasably mounted into a breast pump assembly. In particular, the tube portion 120 may be mounted in a fixed position relative to the breast pump assembly.

The mounting means 160 is formed as a projection 161 on the outer wall 110 of the tube portion 120. The projection 161 extends around the outer wall 110. The projection 161 has a size and profile suitable to locate with a retainer provided in the breast pump assembly. The projection 161 is configured to engage a breast pump element into which the connector is mounted 100, for example a housing. The projection 161 restricts relative axial movement between the connector 100 and the breast pump element.

The mounting means also includes protrusions 162 on the outer wall 110 of the tube portion 120. In this example the protrusions 162 depend from the projection 161, although the protrusions 162 may be provided independently of any projection 161. The protrusions 162, in this example two protrusions, are opposingly arranged around the outer wall 110 of the tube portion 120. The protrusions 162 are configured to engage a breast pump element into which the connector is mounted, for example a housing. The protrusions 162 restrict relative rotational movement between the connector 100 and the breast pump element.

In this way, the mounting means 160 may be configured to restrict one or both of relative axial or rotational movement between the connector 100 and a breast pump element into which the connector 100 is mounted.

In the example shown, the mounting means 160 is integrally formed with the connector 100.

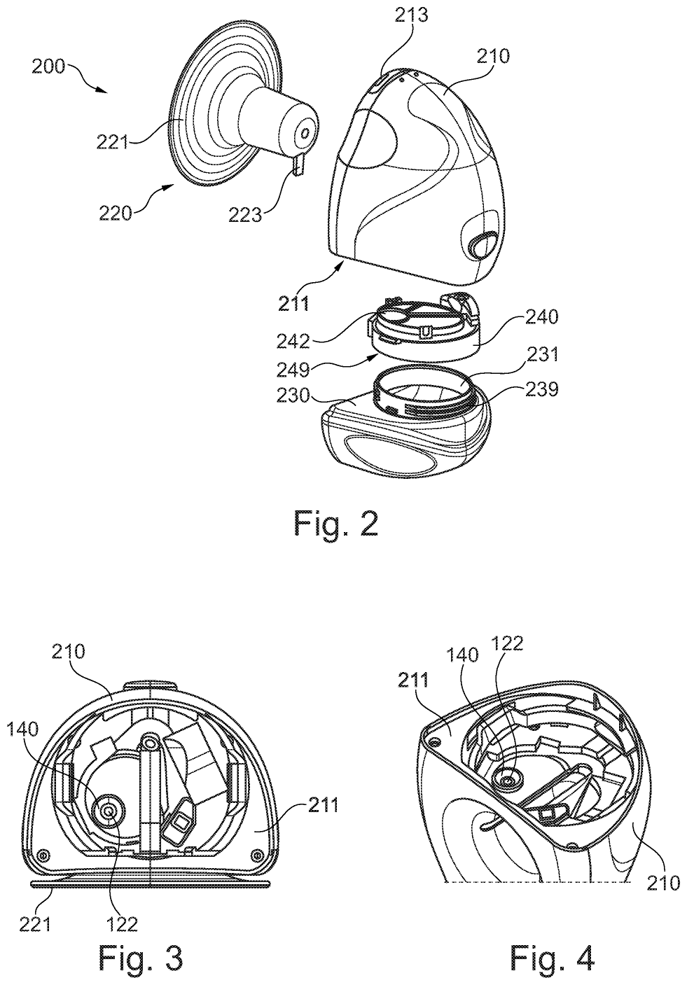

Referring now to FIGS. 2 to 4, there is shown a breast pump assembly 200 including the connector 100 as described with reference to FIG. 1. The assembly 200 includes a housing 210, a horn 220, a container 230 and a cover assembly 240.

The housing 210 includes a negative pressure source mounted within and adapted to provide a suitable negative pressure for expressing milk. Controls 213 to enable a user to operate and adjust the negative pressure are provided on the outside of the housing 210. The housing has a lower housing portion 211 as will be described further below.

The horn 220 is adapted to be mounted within a suitably configured recess in the housing 210. A first portion of the horn 220 is configured to receive a breast for expressing milk. The horn 220 includes a conduit configured to be fluidly connected to negative pressure from the housing 210. In the example shown, the fluid connection is provided through the cover assembly 240, as described in further detail below.

The connector 100 is mounted within the housing 210 so that so that the tube portion 120 is fluidly connected to the negative pressure source.

The connector 100 is mounted to the housing 210 so that its securing member 140 and the first opening 122 are exposed externally relative to the housing 210. The connector 100 is mounted so that the securing member 140 is available for engagement with a breast pump element, in this example a cover assembly 240.

Accordingly, the securing member 140 is mounted in the housing 210 so as to easily and readily engage the cover assembly 240 as it is releasably mounted to the housing 210. In this way, a fluid flow path between the connector 100 and a fluid port 242 on the cover assembly 240 is easily provided by mounting the cover assembly 240 to the housing 210. The user can achieve this without precise alignment or without manually interconnecting tubes or conduits. Consequently, a fluid flow path may be easily provided from the negative pressure source to the cover assembly 240.

The cover assembly 240 is configured to be releasably mounted within the lower housing portion 211. The cover assembly 240 is mounted to the lower housing portion 211 by cooperating inter-engagement means suitably provided on opposing surfaces of the cover assembly 240 and the housing 210. In the example shown, the cover assembly 240 is releasably secured to the housing 210 by rotation relative to the housing 210 so as to engage screw threads.

The cover assembly 240 is configured to be releasably secured to the neck of the container 230. In the example shown, the cover assembly 240 is releasably secured to the housing 230 by rotation relative to the cover assembly 240 so as to engage screw threads 239, 249.

Although screw threads are described for releasably securing components of the breast pump assembly, any suitable engagement or inter-engagement means may be employed as may be known in the art, such as bayonet fittings.

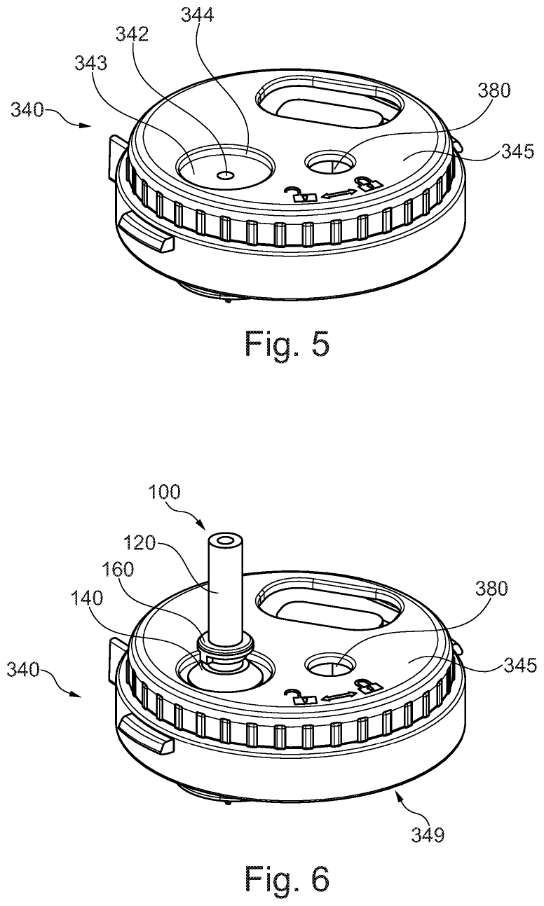

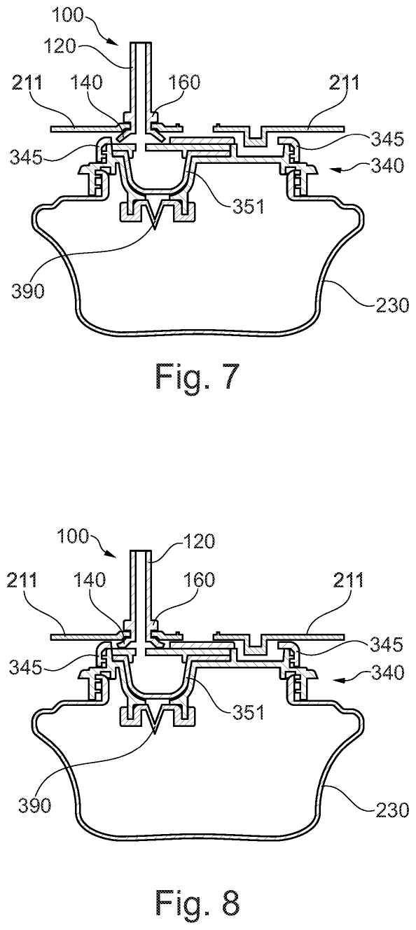

Referring now to FIGS. 5 and 6, there is shown an alternative cover assembly 340 with and without the connector 100 described herein. The cover assembly 340 includes a fluid port 342. The fluid port 342 is provided on an upper member or surface 345 of the cover assembly 340. The fluid port 342 extends through upper member or surface 345 so as to provide a fluid connection to the inner components of the cover assembly 340. In the example shown in FIGS. 7 and 8, the fluid port is fluidly connected to an actuatable diaphragm 351.

The cover assembly 340 includes a receiving portion 343 surrounding the fluid port 342. The receiving portion 343 is configured to encourage a reliable seal when the securing member 140 is urged towards the cover assembly 340 to form a sealing abutment.

In the example shown, the receiving portion 343 includes a polished surface. In alternative examples the receiving portion may include a textured or patterned surface, or any other surface texture or pattern suitable to provide effective sealing abutment when the securing member is urged against the cover assembly.

The fluid port 342 is provided in a recess 344 of the cover assembly 340. In this way the recess 344 provides a locating means on the cover assembly 340 to provide a guide to the securing member 140 as it engages the cover assembly.

In alternative configurations, other locating means may be provided, for example visual guides or physical guides such as depressions in the surface proximal to the fluid port, or ribs or protrusions extending from the surface. As the skilled person will appreciate therefore, the locating means may be any suitable guide to aid the user in quickly and easily assembling elements but without requiring any precise alignment.

In the example shown, the receiving portion 343 is also provided within the recess 344. However, the skilled person will appreciate that one or both of the fluid port 342 and the receiving portion 343 may be provided on any suitable surface of a cover assembly, or other breast pump element, in the absence of a recess. The receiving portion may be provided around any fluid port so as to provide engagement and sealing abutment for the securing member 140.

The cover assembly 340 is configured to convey negative pressure from the housing to an outlet port 380. The outlet port 380 is configured to fluidly connect the cover assembly 340 to a conduit of a horn, such as the horn 210 described with reference to FIGS. 2 to 4. In this way, the cover assembly 340 is configured to fluidly connect the horn to the connector 100, and thereby, to the negative pressure source so as to provide negative pressure for expressing milk.

In use, the cover assembly 340 receives expressed milk from the conduit of the horn. A diaphragm 249, as shown and described with reference to FIGS. 7 and 8, is mounted within the cover assembly 340. The diaphragm 249 is arranged so as to provide a barrier which prevents expressed milk from otherwise entering the housing via the first opening 122 on the connector 100.

The cover assembly 340 is arranged to direct expressed milk from the conduit to the container, such as the container 230 described above, mounted thereto for collection. The cover assembly 340 is provided with suitable one-way valve 390 to the control flow or expulsion of expressed milk into the container 230 while preventing negative pressure being conveyed into the container 230.

Referring now to FIGS. 7 and 8, there are shown aspects of the cover assembly 340 of FIGS. 5 and 6 engaging a housing as described in reference to FIGS. 2 to 4. Accordingly, there is shown the connector 100 and lower housing portion 211 of the housing 210 assembled for use with the cover assembly 340 and container 230.

To assemble the cover assembly 340 and lower housing portion 211 are disposed in a first position, as shown in FIG. 7.

In the first position, the opposing engagement means (not shown) provided respectively on the cover assembly 340 and the housing are initially engaged.

The connector 100 is mounted through an aperture in the lower housing portion 211 so that the securing member 140 and first opening 122 are disposed towards the cover assembly 340. The cover assembly 340 is arranged so that the upper member 345 of the cover assembly is disposed towards the connector 100. In this way, the fluid port 342 is disposed towards the connector 100.

In the first position, the securing member 140 engages the cover assembly 340 and encloses the fluid port 342. A fluid flow path is formed between the first opening 122 and the cover assembly 340.

The annular flange 142 of the securing member 140 has an outer diameter larger than the diameter of the fluid port 342. In this way, as the securing member 140 engages the cover assembly 340 it encloses the fluid port. Stated differently, the connector 100 covers the fluid port.

Further, by using a securing member 140 of a larger diameter, the fluid port is enclosed, or covered, with only a general alignment between the housing and the cover assembly. That is, the securing member 140 is sized such that even if the respective central axes of the annular flange 142 and fluid port are not accurately aligned, the securing member 140 is still able to engage the cover assembly and form a fluid flow path between the first opening 122 and the cover assembly.

When in the first position, a negative pressure is applied within the tube portion 120. The negative pressure resiliently deforms the securing member 140, thereby urging the securing member 140 into sealing abutment with the cover assembly 340. In this way, the connector 100 adopts a second position, as shown in FIG. 8.

The negative pressure applied within the tube portion 120 provides a pressure difference across the securing element 140. A negative pressure is provided within the securing element 140 such that the securing element is urged against the cover assembly 340 by the ambient pressure outside the securing element 140.

Thus, in the second position, the securing element 140 forms a fluid seal with the cover assembly 340. Stated differently, the sealing abutment provided by the resilient deformation of the securing element 140 ensures a fluid-tight seal between the connector 100 and cover assembly 340.

In the second position, the opposing engagement means (not shown) provided respectively on the cover assembly and the housing are fully engaged, thereby securing the cover assembly to the housing. Optionally, the engagement means may be fully engaged before, during or after the securing element is urged into sealing abutment with the cover assembly.

In the second position, the negative pressure within the tube portion 120 can thereby be conveyed through the fluid port 342 to the cover assembly 340. That is, the negative pressure from the negative pressure source within the housing 210 can be conveyed to the cover assembly 340. Thus, with the container 230 and horn conduit fluidly connected to the cover assembly 340, negative pressure from the negative pressure source is conveyed to the horn. The assembly is thus ready for a user to express milk.

In moving from the first position to the second position, the annular flange 142 of the securing element 140 is deformed outwardly. Optionally, in alternative examples, the securing element may be resiliently deformed in any suitable way to provide a sealing abutment. Thus, the securing element may include a folded portion or may include a bellows. Such features may resiliently deform by resiliently deforming one or more the folds. Further alternatively, the securing element may resiliently deform by contracting or collapsing, so as to, for example, deform around a projection or projecting feature on the cover assembly.

The securing element 140 engages the cover assembly 340 in the first position at a perimeter portion 146 of the securing element 140. An area of contact between the securing element 140 and the cover assembly 340 may have a radial width of less than or equal to 1 mm. Thus, due to the annular nature of the annular flange 142 of the securing element, the area of contact in the present example is an annular contact area of radial width of less than or equal to 1 mm.

Upon application of negative pressure within the tube portion 120, that is in moving to the second position, the sealing abutment provides an increased area of contact between the securing element 140 and the cover assembly 340. Thus, the area of contact between the securing element and the cover assembly increases to a radial width of at least 2 mm. That is, the annular contact area increases to a radial width of at least 2 mm.

In this way, the sealing abutment is broad enough to overcome contamination. Due the increased contact area, small particles or ingress of dirt within the sealing abutment, that is between the securing member 140 and breast pump element, will not result in interruption of the seal. The sealing abutment is wide enough to seal around the contamination without causing a leak.

As will be understood by the skilled person, the contact area of the sealing abutment may be modified or tuned by suitable configuration of the securing element. Thus, by adopting a softer material, or suitably configuring the angle or outer diameter of the securing element, the resilient deformation of the securing member may adjusted. In this way, the connector 100 of the invention may be configured according to each breast pump, or elements thereof. The connector may be configured to provide a sealing abutment with different sized ports or using a range of sizes of tube portions. Further, the connector may be operable so resiliently deform in response to a range of negative pressures provided by the negative pressure source while ensuring a reliable seal between connector and breast pump element.

As will be understood by the skilled person, the securing member may be provided in any suitable shape or configuration to enclose, or cover, a range of fluid port shapes. Thus, for example, the connector may be configured to enclose and provide sealing abutment to a range of regular and irregular shapes by configuring the securing member in a corresponding shape.

As will be understood by the skilled person, the securing element may be provided at any angle to the fluid port. Thus the securing element may be configured so that the axis of the tube portion may be aligned at any angle to the axis of the opening of the fluid port. In these arrangements, a sealing abutment may nevertheless be achieved by negative pressure applied within the tube portion by using any suitable orientation of the securing member to engage the breast pump element and enclose the fluid port.

1. A connector suitable for securing to a breast pump element, the connector comprising:
a tube portion with a first opening;
and a securing member, mounted to the first opening, and configured to be engageable with a breast pump element having a fluid port so as to enclose the fluid port and form a fluid flow path between the first opening and the breast pump element; and
wherein the securing member is configured to resiliently deform to be urged into a sealing abutment with the breast pump element when negative pressure is applied within the tube portion.

2. A connector according to claim 1, wherein the securing member comprises an annular flange extending from the first opening.

3. A connector according to claim 2, wherein the annular flange is frustoconical.

4. A connector according to claim 2, wherein the annular flange has a thickness in a range of from 1 mm to 3 mm, preferably in the range of from 1.2 mm to 2.0 mm.

5. A connector according to claim 1, wherein the connector comprises an elastomer.

6. A connector according to claim 1, wherein the sealing abutment provides an area of contact with the breast pump element, and wherein the area of contact increases as the securing member is resiliently deformed.

7. A connector according to claim 2, wherein the annular flange is configured so that a perimeter portion of the annular flange deforms outwardly as the member is resiliently deformed.

8. A connector according to claim 1, wherein the tube portion and the securing member are integrally formed.

9. A connector according to claim 1, wherein the connector comprises a mounting means to mount the connector within a breast pump assembly.

10. A connector according to claim 1, wherein the member is configured to engage with a locating means provided on a breast pump element.

11. A connector according to claim 1, wherein the tube portion is configured to be fluidly connected to a negative pressure source of a breast pump assembly.

12. A breast pump assembly comprising a negative pressure source and a connector of claim 1 mounted to the breast pump assembly and fluidly connected to the negative pressure source.

13. A breast pump assembly according to claim 12, wherein the connector is mounted so as to engage a breast pump element releasably secured to the breast pump assembly.

14. A breast pump assembly according to claim 12, wherein the connector comprises a mounting means to mount the tube portion in a fixed position relative to the breast pump assembly.

15. A breast pump assembly according to claim 14, wherein the mounting means comprises a projection on an outer wall of the tube portion, configured to locate with a retainer provided in the breast pump assembly.

16. A breast pump assembly according to claim 12, configured to be releasably secured to a breast pump element by relative rotation of the breast pump assembly.

17. A breast pump assembly according to claim 16, wherein the breast pump assembly is releasably secured to a breast pump element using a bayonet fitting.

18. A breast pump assembly according to claim 12, wherein the securing member is engaged with a breast pump element so as to enclose a fluid port provided thereon and form a fluid flow path between the first opening and the breast pump element.

19. A breast pump assembly according to claim 18, wherein the breast pump element comprises a polished surface for engaging the securing member.

20. A method of securing a tube portion to a breast pump element, the method comprising:
mounting a securing member to a first opening of a tube portion;
engaging the securing member with a breast pump element having a fluid port provided thereon such that the securing member encloses the fluid port to form a fluid flow path between the first opening and the fluid port; and
applying a negative fluid pressure within the tube portion so as to resiliently deform the securing member and urge the securing member into a sealing abutment with the breast pump element.

21. A method according to claim 20, wherein the step of engaging the securing member with a breast pump element further comprises engaging the securing member with a locating means provided on a breast pump element.

22. A method according to claim 21, wherein the locating means comprises a recess.
